(19) United States
(12) Patent Application Publication
(10) Pub. No.: US 2004/0097951 A1
(43) Pub. Date: May 20, 2004

(54) Measurement instrument for use in orthopaedic surgery

(76) Inventor: Scott J. Steffensmeier, Warsaw, IN (US)

Correspondence Address: BAKER & DANIELS, 111 E. WAYNE STREET, SUITE 800, FORT WAYNE, IN, 46802

(21) Appl. No.: 10/298,634

(22) Filed: Nov. 18, 2002

(51) Int. Cl. A61B 17/58

(52) U.S. Cl. 606/102; 606/86; 606/90

(57) ABSTRACT

A surgical instrument for taking measurements during orthopaedic surgery such as knee replacement procedures. The instrument includes two reference elements which each have an outwardly facing registry surface. The instrument includes a measurement guide which has indicia related to a linear distance between the two registry surfaces and an angular orientation between the two registry surfaces. The reference elements are biased apart and the measurement guide responds contemporaneously with the relative movement of the two reference elements. One of the registry surfaces may be an articulating surface for engagement with a provisional femoral component. The instrument may include a housing from which two elongate members are biasingly projected to support one of the reference elements and the indicia corresponding to the angular orientation of the two registry surfaces may be a second linear measurement corresponding to the distance between the two registry surfaces at a second location.

### BACKGROUND OF THE INVENTION

1. Field of the Invention

The present invention relates to orthopaedic surgery instrumentation and, more specifically, a measurement instrument that can be used in knee replacement procedures.

2. Background of the Invention

In a knee replacement procedure, a worn or damaged knee is replaced with a prosthetic knee joint. One goal of such procedures is to provide a balanced knee joint which demonstrates proper ligament tension through out the full range of motion of the knee joint. One factor in providing such a balanced joint is the proper balancing of the soft tissue by the surgeon. In addition to the orientation and configuration of the implant, one of the factors in providing such a balanced joint is the selection of properly sized implant components.

### SUMMARY OF THE INVENTION

The present invention provides a surgical instrument which assists the surgeon in properly balancing the soft tissue and selecting the properly sized knee prosthesis components.

The invention comprises, in one form thereof, a surgical instrument for taking measurements during orthopaedic surgery wherein the instrument includes two reference elements, each having an outwardly facing registry surface with the two reference elements being substantially disposed between the two outwardly facing registry surfaces. The instrument further includes at least one measurement guide having indicia correlated to a linear distance between the first and second registry surfaces and a measurement correlated to a relative angular orientation of said first and second registry surfaces. In some such instruments, the measurement correlated to the relative angular orientation of the registry surfaces is a linear distance laterally spaced from the location of the first linear distance measurement. The instrument further includes at least one biasing element biasing apart the reference elements and wherein the measurement guide contemporaneously responds to the relative movement of the reference elements as they are biased apart.

In another embodiment of the invention, one of the reference elements may have a registry surface that defines a non-planar articulating surface. The other registry surface may be a planar surface configured for placement parallel to a tibial plateau. The articulating surface of such an instrument may also be shaped in a manner whereby it is configured for continuous, articulating, biased engagement with a femoral component throughout an angular range of motion ranging between flexion and extension positions of the knee.

In yet another embodiment of the invention, the surgical instrument may include a housing and have two elongate members moveably extending from the housing. One of the reference elements may be supported on the elongate members with a separate biasing element engaged with each of the elongate members. Each of the elongate members may also form a measurement guides having indicia thereon to provide information about the linear distance separating the registry surfaces at two different locations.

The invention comprises, in another form thereof, a surgical instrument for taking measurements during orthopaedic surgery, the measurements being taken in reference to an orthopaedic implant, wherein the instrument includes a first substantially planar reference element having an outwardly facing first registry surface and a second reference element having an outwardly facing second registry surface. The second registry surface defines an articulating surface articulatingly engageable with the orthopaedic implant. The instrument also includes at least one measurement guide having indicia correlated to a relative position of the first registry surface with respect to the second registry surface and at least one biasing element biasing the first and second reference elements apart from each other. The at least one measurement guide contemporaneously responds to relative movement of the first and second reference elements as the first and second elements are biased apart.

The invention comprises, in yet another form thereof, a surgical instrument for taking measurements during orthopaedic surgery, the measurements being taken in reference to an orthopaedic implant, wherein the instrument includes a first reference element having a first outwardly facing registry surface and a second reference element moveable relative to said first reference element along a longitudinal direction and having a second outwardly facing registry surface. The instrument also includes at least one measurement guide having indicia correlated to a relative position of the first registry surface with respect to the second registry surface and at least one biasing element longitudinally biasing the second reference element away from the first reference element. The at least one measurement guide contemporaneously responds to relative movement of the first and second reference elements as the first and second elements are biased apart. In one embodiment of such an instrument, the at least one measurement guide includes at least two measurement guides wherein each of the measurement guides have indicia providing information correlated to a linear, substantially longitudinal, distance between the first and second registry planes at first and second measured locations. The first and second measured locations are spaced apart in a lateral direction substantially perpendicular to the longitudinal direction.

An advantage of the present invention is that by including a biasing element which biases the registry surfaces apart and a measurement guide which contemporaneously responds to the relative movement of the reference elements as they are biased apart, the instrument can be used to obtain contemporaneous readings on the parameters measured by the instrument.

Another advantage of the invention is that for those embodiments which have as one of the registry surfaces an articulating surface, the instrument may be configured to provide measurements as a provisional implant articulates against the registry surface. For example, it is anticipated that it will be useful for the articulating surface to be configured to receive the provisional femoral component when conducting a knee replacement procedure whereby a surgeon may leave the instrument in place while moving the patient's leg between flexion and extension. Moreover, the measurement guide, by providing readings on the measured parameters throughout the range of motion through which the limb is moved while the limb is being moved, has the potential to provide the surgeon with valuable information on the soft tissue balance and the proper size to select for an implant component at many different locations throughout the range of motion.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is an exploded and partial cutaway view of one embodiment of an instrument of the present invention.

FIG. 2 is a view of the instrument being used with a knee in extension and with a tibial plate.

FIG. 3 is a view of the instrument being used with a knee in flexion and without a tibial plate.

FIG. 4 is a view of an instrument and a joint with an exaggerated imbalance.

Corresponding reference characters indicate corresponding parts throughout the several views. Although the drawings represent an embodiment of the present invention, the drawings are not necessarily to scale and certain features may be exaggerated in order to better illustrate or explain the present invention.

The exemplification set out herein illustrates an embodiment of the invention, in one form, and is not intended to be an exhaustive illustration of the invention or to be construed as limiting the scope of the invention to the form disclosed.

### DESCRIPTION OF THE PRESENT INVENTION

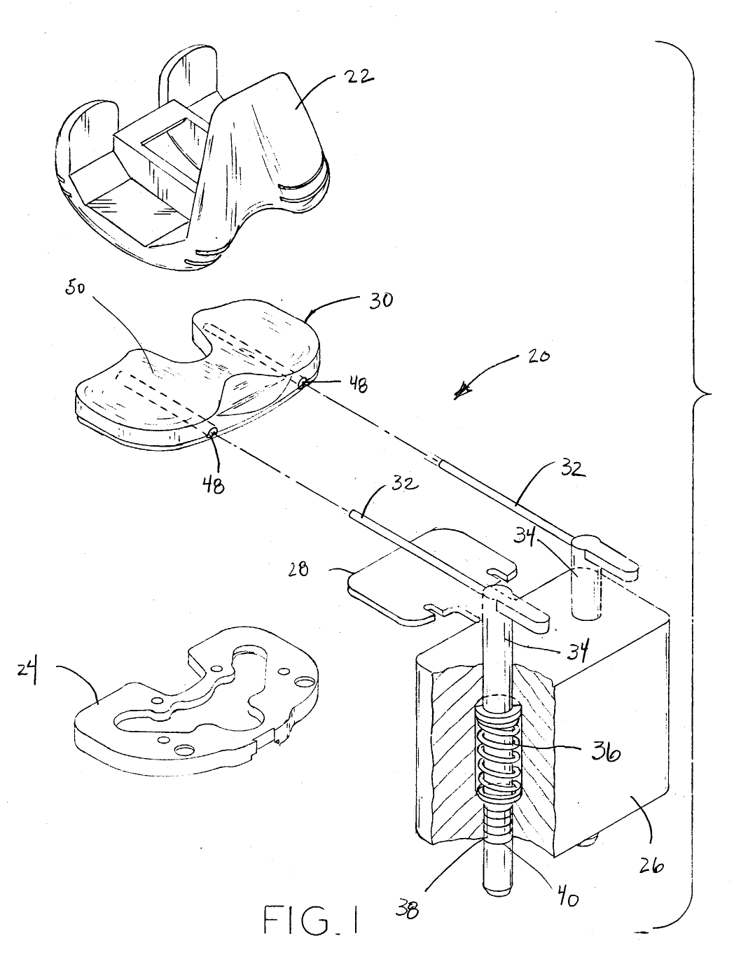

An instrument 20 in accordance with the present invention is shown in FIG. 1. Also shown in FIG. 1 is a femoral provisional component 22 and a tibial sizing plate 24. The femoral provisional 22 and tibial sizing plate 24 are conventional components known in the art.

The instrument 20 includes a main housing 26, a first reference element 28 and a second reference element 30. The second reference element is mounted on mounting members 32 which, in the illustrated embodiment, are cylindrical rods. The mounting members 32 extend outwardly from posts 34. As can be seen in partially cutaway FIG. 1, the posts 34 are biased upwardly in the direction of the femoral provisional by a biasing element 36 which, in the illustrated embodiment, is a helical spring. Alternative biasing elements may also be used. The lower portion of the elongate members or posts 34 form a measurement guide 38 having indicia 40 located thereon. Housing 26, first and second reference elements 28, 30, mounting members 32 and posts 34 may be manufactured of stainless steel or other suitable materials.

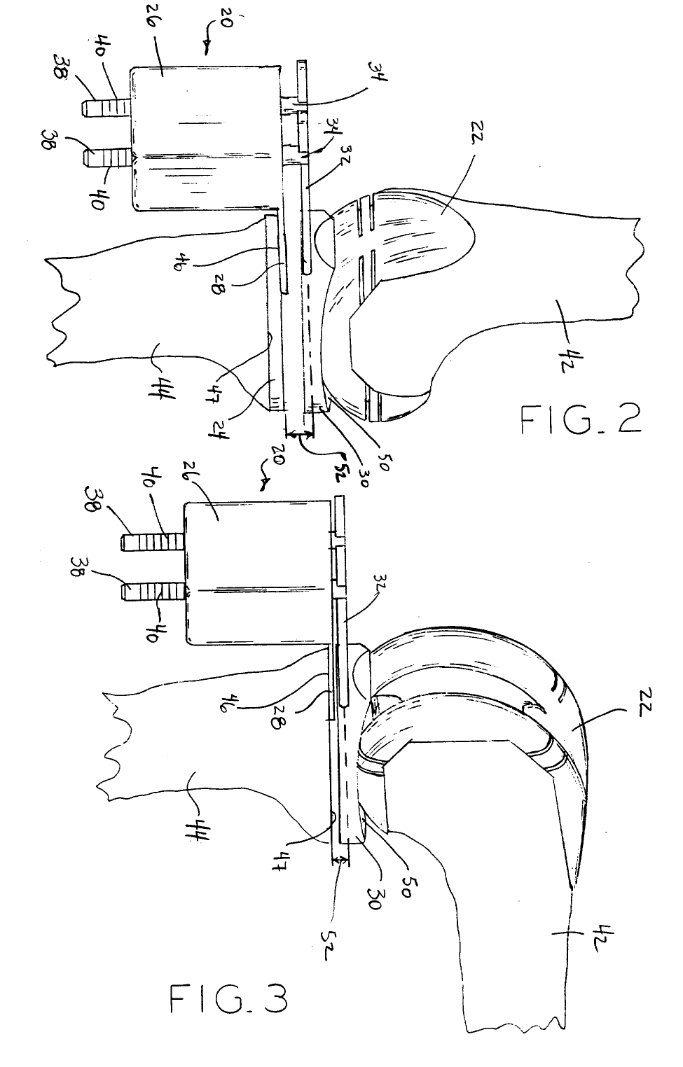

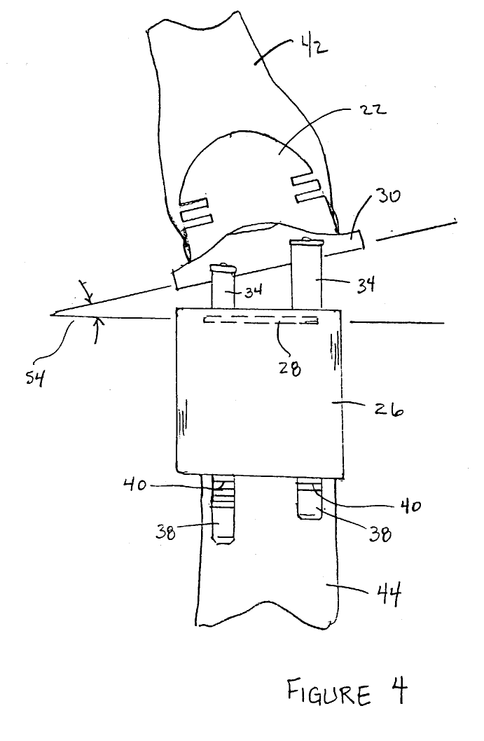

The use of instrument 20 is best seen in FIGS. 2 and 3. FIGS. 2 and 3 illustrate a femur 42 and a tibia 44 where the distal femur and the proximal tibia have already been prepared by the surgeon in a manner which is well known in the art. FIG. 2 illustrates a femur 42 and tibia 44 where the knee joint is in extension. In FIG. 2, a tibial sizing plate 24 has been positioned on the tibial plateau 47 which has been formed on the proximal tibia by the surgeon. In FIG. 2, the registry surface 46 located on first reference element 28 is placed in registry with the upper surface of the tibial sizing plate.

The second reference element 30, has openings 48 which receive rods 32 to mount the second reference element 30 thereon. Reference element 30 also includes a registry surface 50 which, in the illustrated embodiment, is an articulating surface engageable with a provisional femoral 22. When instrument 20 is assembled, registry surfaces 46 and 50 face in opposite, outwardly directions. The biasing elements 36 act on posts 34 to bias the registry surfaces 46, 50 away from each other. Consequently, when the first and second reference elements are positioned in the spacing between the tibia and femur as illustrated in FIGS. 2 and 3, the registry surfaces 46, 50 are biased outwardly into engagement with a surface on the tibia and on the femur respectively.

As the two registry surfaces 46, 50 are positioned at different relative positions, a measured distance 52 between the two registry surfaces changes resulting in a corresponding change in the designated indicia 40. The relative angular position of registry surfaces 46, 50 may also change as the two registry surfaces 46, 50 are positioned at different relative positions. In the illustrated embodiment, the designated indicia 40 corresponds to the indicia located nearest the housing 26 from which the measurement guides 38 extend. Although not illustrated in the Figures, indicia 40 may take the form of bands having, for example, numeric values such as a band labelled and corresponding to a gap 52 having a value of 4 mm. Alternatively, indicia 40 may take the form of colored bands wherein red corresponds to a particular sized gap 52 and the implant components may also be color coded whereby the surgeon may readily select a “red” implant to go with a “red” gap.

The illustrated embodiment has two separate spring biased posts 34 which support the femoral reference element 30. These posts 34 are independently moveable and the indicia 40 of the measurement guides 38 may provide different readings if the registry surfaces 46, 50 are positioned at a relative angle to each other. One situation where the ability of the two measurement guides 38 to provide different readings would be beneficial is where the soft tissue of the knee joint is unbalanced. When the soft tissue is balanced, on the other hand, the two measurement guides 34 should designate the same indicia 40.

Thus, the illustrated embodiment utilizes two elongate members, i.e., posts 34, which extend from the housing 26 to measure a linear distance 52 which is directly related to the linear distance between the two registry surfaces 46, 50 at two different spaced apart locations. This not only provides a means of determining the size of the articulating surface component which will be implanted by directly measuring the gap between tibia and femur, but also provides a means of measuring the relative angular orientation between the two registry surfaces 46, 50 along one direction. In the illustrated embodiment, when the instrument 20 is being used as illustrated in FIGS. 2 and 3, the two locations of the posts 34 are spaced laterally apart in the lateral/medial direction but are located in the same anterior/posterior plane. Thus, the measurement of this one relative angular orientation between the two registry surfaces is accomplished using two spaced apart linear measurements in the illustrated embodiment.

FIG. 4 illustrates an instrument 20 and how it may be used with a joint having an imbalance. The imbalance depicted in FIG. 4 has been exaggerated to more clearly illustrate the operation of instrument 20. As shown in FIG. 4, the registry surfaces 46, 50 are at a relative angular orientation 54. The measurement guides 38 designate different indicia 40 to reflect the different linear distances between registry surfaces 46, 50 at the two different measured locations which in turn reflects the relative angular orientation between the two registry surfaces 46, 50.

As best seen in FIG. 1, the reference element 30 defining articulating registry surface 50 includes two openings 48 through which the mounting members 32 are inserted to thereby support reference element 30 on the instrument 20. The lower reference element 28 is fixed in position to the housing 26 from which the posts 34 extend to support the other reference element 30. Consequently, reference element 30 must tilt relative to housing 26 for the reference element 30 to change its angular orientation relative to reference element 28 and openings 48 are sufficiently larger than mounting members 32 for the reference element 30 to tilt. Alternatively, openings 48 may be shaped in a more slot like manner to permit adjustments in the angular orientation between the two registry surfaces.

In FIG. 2, the registry surface 46 is engaged with the upper surface of tibial sizing plate 24. It is not necessary to use a tibial sizing plate with instrument 20 and FIG. 3 illustrates the registry surface 46 in direct engagement with the tibial plateau 47. In both of the situations illustrated in FIGS. 2 and 3, the registry surface 46 is positioned substantially parallel to the tibial plateau 47.

Illustrated articulating surface 50 has been shaped to have a configuration similar to that of the articulating surface formed by the insert which is implanted as part of the knee prosthesis and against which the femoral component bears in an articulating manner. This allows the femoral provisional 22 to engage and articulate against registry surface 50 as the surgeon moves the patient's leg from extension (as exemplified in FIG. 2) to flexion (as exemplified in FIG. 3) in the same fashion as the implanted femoral component will engage and articulate against the articular surface insert, i.e., the component forming the articulating surface of the implanted knee prosthesis.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains. Accordingly, the scope of the invention should be determined not by the illustrated embodiments but by the following claims and their legal equivalents.

What is claimed is:

1. A surgical instrument for taking measurements during orthopaedic surgery, said instrument comprising:
a first reference element having a first outwardly facing registry surface;
a second reference element having a second outwardly facing registry surface, said first and second reference elements being substantially disposed in a space located between said first and second outwardly facing registry surfaces;
at least one measurement guide having indicia correlated to at least one linear distance between the first and second registry surfaces at a first location and at least one measurement correlated to a relative angular orientation of said first and second registry surfaces; and
at least one biasing element biasing said first and second reference elements apart from each other, said measurement guide contemporaneously responding to relative movement of said first and second reference elements as said first and second elements are biased apart.

2. The surgical instrument of claim 1 wherein said at least one measurement correlated to the relative angular orientation of said first and second registry surfaces comprises a second linear distance between the first and second registry surfaces at a second location spaced from said first location.

3. The surgical instrument of claim 1 wherein said second reference element is removable from said instrument.

4. The surgical instrument of claim 1 wherein said second registry surface defines a non-planar articulating surface.

5. The surgical instrument of claim 4 wherein said first registry surface is a planar surface configured for placement parallel to a tibial plateau and said articulating surface is configured for continuous, articulating, biased engagement with a femoral component throughout an angular range of motion ranging between flexion and extension positions.

6. The surgical instrument of claim 1 further comprising a housing and two elongate members moveably extending from said housing; said second reference element supported on said two elongate members; said at least one biasing element comprising two biasing elements, each of said biasing elements biasingly engaged with one of said elongate members; and wherein said at least one measurement guide comprises two measurement guides, each of said measurement guides comprising indicia located on a respective one of said elongate members.

7. The instrument of claim 5 wherein said first reference element extends from and is affixed to said housing.

8. A surgical instrument for taking measurements during orthopaedic surgery, the measurements being taken in reference to an orthopaedic implant, said instrument comprising:
a first substantially planar reference element having an outwardly facing first registry surface;
a second reference element having an outwardly facing second registry surface, said second registry surface defining an articulating surface articulatingly engageable with the orthopaedic implant;
at least one measurement guide having indicia correlated to a relative position of said first registry surface with respect to said second registry surface; and
at least one biasing element biasing said first and second reference elements apart from each other, said at least one measurement guide contemporaneously responding to relative movement of said first and second reference elements as said first and second elements are biased apart.

9. The surgical instrument of claim 8 wherein said second reference element is removable from said instrument.

10. The surgical instrument of claim 8 wherein said first registry surface is a planar surface configured for placement parallel to a tibial plateau and said articulating surface is configured for continuous, articulating, biased engagement with a femoral component throughout an angular range of motion ranging between flexion and extension positions.

11. The surgical instrument of claim 8 further comprising a housing and two elongate members moveably extending from said housing; said second reference element supported on said two elongate members; said at least one biasing element comprising two biasing elements, each of said biasing elements biasingly engaged with one of said elongate members; and wherein said at least one measurement guide comprises two measurement guides, each of said measurement guides comprising indicia located on a respective one of said elongate members.

12. The instrument of claim 11 wherein said first reference element extends from and is affixed to said housing.

13. The instrument of claim 8 wherein said indicia of said at least one measurement guide comprises indicia correlated to a linear distance between said first and second registry surfaces and to a relative angular orientation of said first and second registry surfaces.

14. A surgical instrument for taking measurements during orthopaedic surgery, the measurements being taken in reference to an orthopaedic implant, said instrument comprising:
a first reference element having a first outwardly facing registry surface;
a second reference element moveable relative to said first reference element along a longitudinal direction, said second reference element having a second outwardly facing registry surface;
at least one measurement guide having indicia correlated to a relative position of said first registry surface with respect to said second registry surface; and
at least one biasing element longitudinally biasing said second reference element away from said first reference element, said at least one measurement guide contemporaneously responding to relative movement of said first and second reference elements as said first and second elements are biased apart.

15. The surgical instrument of claim 14 wherein said at least one measurement guide comprises at least two measurement guides; said measurement guides each having indicia providing information correlated to a linear, substantially longitudinal, distance between said first and second registry planes at first and second measured locations, said first and second measured locations being spaced apart in a lateral direction substantially perpendicular to the longitudinal direction.

16. The surgical instrument of claim 15 further comprising a housing and two elongate members moveably extending from said housing; said second reference element supported on said two elongate members; said at least one biasing element comprising two biasing elements, each of said biasing elements biasingly engaged with one of said elongate members; and wherein each of said measurement guides comprises indicia located on a respective one of said elongate members.

17. The surgical instrument of claim 14 wherein said second reference element is removable from said instrument.

18. The surgical instrument of claim 14 wherein said first registry surface is a planar surface configured for placement parallel to a tibial plateau and said second registry surface is an articulating surface configured for continuous, articulating, biased engagement with a femoral component throughout an angular range of motion ranging between flexion and extension positions.
